(19) United States
(12) Patent Application Publication
(10) Pub. No.: US 2012/0245386 A1
(43) Pub. Date: Sep. 27, 2012

(54) SYNTHESIS OF TETRABUTYLAMMONIUM BIS(FLUOROSULFONYL)IMIDE AND RELATED SALTS

(75) Inventor: Martin R. Johnson, Piedmont, CA (US)

(73) Assignee: Trinapco, Inc., Oakland, CA (US)

(21) Appl. No.: 13/426,331

(22) Filed: Mar. 21, 2012

Related U.S. Application Data
(60) Provisional application No. 61/465,647, filed on Mar. 21, 2011.

(51) Int. Cl. C07C 311/48

(52) U.S. Cl. 562/823

(57) ABSTRACT

The present invention is directed to methods comprising adding ammonia to a sulfuryl fluoride solution to form the anion of bis(fluorosulfonyl)amine under conditions well suited for large-scale production. The bis(fluorosulfonyl)amine so produced can be isolated by methods described in the prior art, or isolated as an organic ion pair, such as an alkylammonium solid salt, or as an ionic liquid.

### CROSS-REFERENCE TO RELATED APPLICATIONS

This application claims the benefit of the filing date of U.S. Provisional Appl. No. 61/465,647, filed Mar. 21, 2011, which is incorporated by reference herein in its entirety.

### BACKGROUND OF THE INVENTION

1. Field of the Invention

The present invention is directed to nonobvious improvements in the preparation of tetrabutylammonium bis(fluorosulfonyl)imide, [Bu₄N]⁺[(FSO₂)₂N]⁻, and related salts. 2. Background

Compounds containing bis(fluorosulfonyl)imide [(FSO₂)₂N]⁻, are useful, for example, as Lewis acid catalysts, ion transport agents, in the fields of organic compound syntheses, electrolytes and the like.

Various methods for synthesizing bis(fluorosulfonyl)amine, and related compounds have been proposed (see, e.g., Ruff, Inorg. Chem. 4(10):1446 (1965); Ruff, Inorg. Synth. XI:138 (William, ed., McGraw-Hill Book Co., 1968); Vij et al., Coord. Chem. Rev. 158:413 (1997); Krumm et al., Inorg. Chem. 37:6295 (1998); Beran et al., Z. Anorg. Allg. Chem. 631:55 (2005); U.S. Pat. No. 5,723,664; and U.S. Pat. No. 5,874,616; DE Patent No. 1 199 244). However, these methods may not be appropriate for industrial scale production because they provide low yields, require the formation of dangerous intermediates, and/or require corrosive and/or expensive starting materials.

U.S. Pat. No. 5,874,616 describes the addition of a fourfold excess of F₃CSO₂NH₂ to SO₂F₂/Et₃N at −30° C. to produce F₃CSO₂NHSO₂F in 55% yield. It also describes the addition of anhydrous NH₃ to F₅C₂SO₂F/Et₃N to give F₅C₂SO₂NH₂, in an example of slow addition of NH₃ to a perfluoroalkyl sulfonamide. Also described is the synthesis of [CF₃(CF₂)₃SO₂]₂NH from CF₃(CF₂)₃SO₂F and NH₃/Et₃N, after heating to 90° C.; similarly, the perfluoro analog was obtained.

An important advance in obtaining [(FSO₂)₂N]⁻ and its salts was disclosed by Morinaka (US2012/0028067 A1), which is incorporated by reference herein in its entirety, who treated a solution of SO₂F₂ in acetonitrile with ammonia, in the presence of an organic base, to obtain [(FSO₂)₂N]⁻ in high isolated yields as various metal salts. By use of elevated pressure conditions, Morinaka was able to contain SO₂F₂ and thereby allow it to react with the ammonia at high concentration. Those skilled in the art will recognize that Morinaka's examples 1-4 use reactor pressures in excess of 3 atmospheres. The elevated pressure conditions described by Morinaka are problematic for commercial scale synthesis of (FSO₂)₂N] salts; since large scale synthesis would require large pressurized vessels. However, large pressurized reactors are substantially more expensive than reactors designed for use at atmospheric pressure and below. Additionally, there are safety issues which arise from the handling of SO₂F₂, as it is highly toxic and completely undetectable by the senses, or by common forms of measurement. Leaks can be fatal to the operator. Those skilled in the art will also recognize that the described examples of Morinaka give substantial amounts of solids in the crude pot liquor, necessitating introduction of an aqueous waste stream into the process. For industrial scale production of (FSO₂)₂N]⁻ salts, operation at or below atmospheric pressure, and under conditions which give an all-liquid pot liquor, are much preferred.

### BRIEF SUMMARY OF THE INVENTION

I have found that the addition of ammonia (NH₃) to a solution of sulfuryl fluoride (SO₂F₂), can be accomplished at ambient pressure and below, to give very good yields of product, with reactor loads in excess of 1 molal. Using these conditions, purified product can be isolated in 95% yield as a tetrabutylammonium salt, [Bu₄N]⁺[(FSO₂)₂N]⁻.

In an embodiment of the invention, gaseous NH₃ is infused into the head space above a stirred solution of SO₂F₂, and/or slowly added as a solution of NH₃ in a solvent. Alternatively, NH₃ can be added as an ammonium salt, provided a base is present in the SO₂F₂ solution or separately added to the solution. For example, an ammonium salt can be added as a solid, a dissolved solid, an ionic liquid, and/or as a dissolved ionic liquid. These liquid-addition embodiments offer some advantage, in that the accumulation of solid deposits on the walls of the reactor can be mitigated by subsurface introduction of the NH₃.

Preferably, the complete consumption of SO₂F₂ is the endpoint of the reaction, which can be determined by a decrease in reactor pressure to a value approaching the vapor pressure of the solvent system. However, the reaction may be halted at any time and the unreacted SO₂F₂ vented and recovered, if desired. Also, air or an inert gas may be introduced to the vessel after the addition of one or more reagents is complete, in order to maintain the reactor pressure close to atmospheric.

Acetonitrile is a preferred solvent; propionitrile is also preferred if dilute injection is used as described below. Tertiary amides are also preferred in some embodiments.

There are two organic bases which are preferred for ambient and subambient pressure operation: tetramethylethylenediamine (“TMEDA”) and tetramethylpropylenediamine (“TMPDA”). These two bases offer the highest reactor loads, produce monophasic pot liquors, give concentrated liquors which are water-soluble when warm, and have vapor pressures below that of acetonitrile and propionitrile. They also have moderate boiling points which allow for their removal after deprotonation. Higher peralkylated polyamines may also be used to good advantage.

The reactor contents are vigorously agitated or stirred in order to prevent formation of side products.

High reactor loads can be accomplished by the introduction of SO₂F₂ gas in a pressure-dependent fashion (a “pressure gate”), as it is consumed by the reaction, to maintain a specific reactor pressure.

With acetonitrile and TMPDA as solvent and base respectively, reactor loads of 1.1 molal can be achieved, providing about 95% isolated yield. Higher loads can be employed, but impurities begin to form above about 1.1 molal.

If the NH₃ is introduced as a gas, it can be introduced into the head space above the liquid. The NH₃ gas must be introduced slowly, over a period of two or more hours, even with vigorous agitation.

If the NH₃ is introduced as a gas into the head space, solids may accumulate in the head space, resulting in reduced yield. This can be prevented by continuous irrigation or wetting of the entire interior surface of the reactor with the pot liquor.

Gaseous NH₃ may be directly injected into the liquid reactor contents at depth, provided that the NH₃ is diluted prior to injection with purified SO₂F₂ (from the head space above the reactor contents) to a ratio not greater than about 2.5% p/p (i.e., about 19 Torr partial pressure of NH₃ for a reaction performed at 760 Torr pressure). This can greatly reduce, or completely eliminate, the accumulation of solids on the interior surfaces of the reactor.

### DETAILED DESCRIPTION OF THE INVENTION

The present invention is directed to nonobvious improvements in the preparation of tetrabutylammonium bis(fluorosulfonyl)imide, [Bu₄N]⁺[(FSO₂)₂N]⁻. Reactor pressures at or just below atmospheric are preferred; however, a reactor pressure well below atmospheric can likewise be employed within the scope of the present invention. In practice, a reactor pressure well below atmospheric results in a reduced concentration of SO₂F₂ and the formation of increased side products and/or a longer addition time. Within the scope of the invention, the reactor may be charged with SO₂F₂ by sparging of the reactor with SO₂F₂ until all other gases are removed, and the reactor is saturated with SO₂F₂. Although this method requires that gaseous SO₂F₂ be collected as an effluent, it has the advantage of eliminating the use of reduced pressure at the start of the reaction. If (after all reagents are added) an inert gas is introduced to maintain atmospheric pressure at the end of the reaction, the entire reaction may be conducted at atmospheric pressure.

Precision control of SO₂F₂ and/or NH₃ introduction can be maintained using, e.g., mass flow controllers, caliper gauges, and the like. In some embodiments, the rate of NH₃ addition (and/or SO₂F₂ addition) is controlled by internal reactor pressure, reactor temperature, or other variable conditions.

SO₂F₂ is highly toxic and completely odorless and colorless. Thus, significant precaution must be used when handling this substance. All reactions should be conducted in areas having sufficient ventilation. On the lab scale, this means all reactions must be conducted inside a fume hood, as well as post-reaction manipulations of the products. On the industrial scale, proper ventilation should be designed and proper safety measures followed. A principal advantage of this invention is the increased safety of the process.

The reaction of NH₃ with a solution of SO₂F₂ is highly exothermic and extremely rapid, and the rate of NH₃ addition should be carefully controlled. In preferred embodiments, the NH₃ is slowly added over a course of at least 90 minutes, or 2 hours or longer to a vigorously stirred SO₂F₂ solution. The rate of addition is typically regulated by the rise in temperature above a starting temperature. In some embodiments, the rise in temperature from the starting static temperature is maintained at ±5° C. or less, and more preferably ±2° C. or less during the addition of NH₃. Effective cooling of the reactor is required to remove the heat of reaction. This is especially important at large scale.

Dissolution of SO₂F₂ can be measured by comparison of the static vapor pressure in the reactor (of the SO₂F₂/solvent blend) with the static vapor pressure of pure SO₂F₂ under the same conditions. Additionally, the solvent may display exothermic mixing with SO₂F₂.

The theoretical molar ratio of NH₃ to SO₂F₂ is 1:2. Practically, a molar ratio of 1.008:2 has been employed, which provided a 95% yield. Larger mole ratios can be employed but offer no advantage, and increase the likelihood of byproduct formation.

The order and rate of addition of NH₃ and SO₂F₂ can be varied, within limits. There must at all times be a large molar excess of SO₂F₂ in the reactor. For example, NH₃ can be added at a continuous rate to a reactor charged with SO₂F₂ to 760 Torr, and additional SO₂F₂ added portion-wise in a pressure-dependent fashion using, e.g., a gated valve. In a preferred embodiment, both reagents are introduced simultaneously at a controlled rate over, e.g., two to four hours for a two gallon reactor. For example, additional SO₂F₂ can be added when the reactor pressure drops below, e.g., 760 torr.

The rate of NH₃ addition can be varied as a function of the degree of agitation of the reactor contents: better mixing in the reactor allows for more rapid addition of NH₃. The rate of NH₃ addition should be controlled to reduce the formation of byproducts. For a two-gallon reactor with maximum agitation, a two-hour addition time was sufficient to provide yield of 90% or greater. While nonetheless within the scope of the invention, under similar conditions adding the NH₃ at a constant rate over one hour resulted in reduced yield and formation of higher amounts of insoluble byproducts.

The base (“B”) can be a tertiary alkylamine. Preferably, the amine base is capable of remaining dissolved in the aprotic polar solvent as a salt with the components (i.e., [BHₘ]^{x+}([(FSO₂)₂N]⁻)ₙ and BH⁻F⁻, where x, m, and n are independently integers from 1 to 4). Exemplary non-reactive bases suitable for use with the present invention have largely been outlined by Morinaka (triethylamine, tripropylamine, 4-N,N-dimethylaminopyridine, 1,8-diazabicyclo[5.4.0]undec-7-ene (“DBU”), 1,5-diazabicyclo[4.3.0]non-5-ene (“DBN”), TMEDA, TMPDA (not mentioned by Morinaka), higher peralkylated polyamines, and combinations thereof Pyridine can also be employed as the base, however, with lower yield (e.g., about 20%). Most preferably, the base is either TMEDA or TMPDA, or a combination thereof These two bases enable the highest reactor loads, are inexpensive, and can be recycled from the process waste. TMPDA in particular gives a monophasic pot liquor, so that issues of solid separation from the reactor contents do not arise. Further, these bases are water miscible (and thus create fewer problems when water is employed as a separating agent), and their boiling points are greater than for some solvents, i.e., acetonitrile (thus, the endpoint reactor pressure is unaffected, which is not the case with bases such as trimethylamine that have a lower boiling point). TMPDA and TMEDA as process bases yield concentrates which are water-soluble when warm, and have low melting points. These two bases also have moderate boiling points which allow for their removal after deprotonation.

Acceptable solvents include ethers (e.g., diethylether, diisopropylether, and the like), nitriles (e.g., acetonitrile, butyronitrile, and the like), esters (e.g., ethyl acetate and the like) halocarbons (e.g., dichloromethane and the like), and tertiary amides (e.g., N,N-dimethylacetamide (DMA), N-methylpyrrolidinone (NMP), tetramethylurea (TMU), dimethylpropyleneurea (DMPU), and the like). Sulfoxides such as dimethylsulfoxide should be avoided; their combination with SO₂F₂ is very dangerous. Solvents with higher polarity are more preferred. At room temperature and pressure, a SO₂F₂ concentration of about 0.4 molal can be readily achieved in acetonitrile/TMPDA. The SO₂F₂ concentration can be maintained by use of, e.g., pressure-gated addition during the course of the reaction, thereby increasing the reactor load. The SO₂F₂ may be added to the head space above the liquid, or more preferably, into the liquid via a dip tube with a disperser.

The equivalent ratio of base to SO₂F₂ in theory is not less than 3:2. In practice, I have found with TMPDA that a mole ratio of 1.03:1 (equivalent ratio 2.06:1 or about 4:2) gave a 95% yield. This ratio can be reduced to a level approaching theoretical without substantially affecting the yield, provided the base is stronger than NH₃, as is the case with both of the nitrogen atoms in TMPDA.

The amount of solvent required is a function of the solubility of the products (especially fluorides) in the solvent. Low polarity solvents cannot give reactor loads of 1 molal without deposition of solid in the reactor; most give much lower loads. More polar solvents can give reactor loads in excess of 1 molal. With acetonitrile and TMPDA as solvent and base respectively, reactor loads of 1.1 molal were achieved without solid formation, providing a 95% isolated yield. Higher loads were employed, but impurities began to form above about 1.1 molal in this solvent/base combination. DMA, NMP, TMU, DMPU and other amide-containing solvent systems can give even higher loads.

Although any temperature between −10° C. and +40° C. is acceptable, temperatures above 0° C. are preferred, more preferably temperatures of 20° C. to 40° C., most preferably temperatures of 23° C. to 28° C. Decreasing the temperature reduces the rate of product formation, whereas increasing the temperature decreases the concentration of dissolved gas in the reactor and increases byproduct formation. Additionally the reactor solution discolors at temperatures above about 35° C.

Solid deposition on the interior surfaces of the reactor was frequently observed in acetonitrile solvent, and is undesireable. This problem is particularly acute with the more volatile bases. Solids form wherever interior surfaces are not liquid-wet. One way to prevent solid formation is constant irrigation of the reactor interior surfaces. This may be accomplished by maximum agitation and a full reactor. Thus, in some embodiments, a reactor is filled to at least 90% of its volume, at least 95% of its volume, or at least 98% of its volume. The function of the near-full capacity is not just to increase reactor load, but to provide irrigation of the interior surfaces of the reactor. Within the scope of the invention, other forms of reactor interior surface irrigation (i.e., spray jets, etc.) may be employed at lower fill levels. In order to maintain proper mixing and irrigation, multiple stir paddles can also be utilized; for example, placing one of the reactor stir paddles near the surface, and stirring as fast as possible. Sufficient agitation and/or irrigation to prevent solid deposition can significantly affect product yield. For example, yields in the range of about 65% to about 80% were obtained even with some solid formation. When proper agitation and irrigation was used to eliminate solid formation, the isolated yield increased to 95%.

Solid deposition on the interior surfaces of the reactor can also be mitigated by direct injection of the NH₃ gas into the liquid contents of the reactor. However, injection of pure NH₃ can cause predominant formation of side products. This can be avoided by dilution of NH₃ with purified SO₂F₂ from the reactor head space (“dilute injection”). Dilution factors of greater than about 90% p/p are preferable; more preferably, greater than about 95% p/p; most preferably, greater than about 97.5% p/p. A 97.5% p/p dilution corresponds to a NH₃ partial pressure of 19 Torr for a reaction run at 760 Torr. Dilution factors can be measured by, e.g., infrared spectroscopy, and controlled by, e.g., mass flow regulators or caliper valves. In this embodiment of the invention, the injected gas is dispersed into bubbles sufficiently fine that the NH₃ contained therein completely reacts with dissolved SO₂F₂ prior to reaching the head space above the surface of the liquid.

Dilute injection can require the SO₂F₂ diluent to be purified. The reactor head space can contain, in addition to the predominant SO₂F₂ vapor, solvent and organic base vapors. Both solvent and organic base vapors must be removed (“scrubbed”) from the diluent gas prior to introduction of NH₃. Any form of solvation of SO₂F₂ will cause a reaction to take place with NH₃, whereas the free gases do not react under ambient conditions.

Scrubbing can be accomplished with a condenser. In this embodiment of the invention, organic bases which are less volatile are more preferred. Solvents with higher boiling points are likewise more preferred. In this embodiment of the invention, the tertiary amide solvents (such as NMP, TMU, and DMPU), as well as TMEDA and TMPDA bases, are particularly well suited for scrubbing due to their lower vapor pressures, compared to acetonitrile, propionitrile, and the lower alkylamine bases such as triethylamine and trimethylamine. By use of higher boiling solvents and bases, higher condenser temperatures can be used, and the diluent gas can be more fully scrubbed.

Scrubbing temperatures should be sufficiently cold. For example, if acetonitrile solvent is used, the condenser temperature can be as low as −47° C., just above the freezing point of acetonitrile (−48° C.) At this temperature, the vapor pressure of acetonitrile is about 0.5 Torr, or 0.07% p/p at 750 Torr operating pressure. Gas flow through the condenser should be low enough that thermodynamic equilibrium is reached at the outlet, i.e., the scrubbing is complete. Absolute gas flow rates are dependent on the scale of the reaction. The scrubbed SO₂F₂ can then be warmed to, e.g., a temperature above the boiling point of NH₃, prior to dilute injection.

By use of the above improvements, scaleup to the metric ton level can be achieved at a minimum cost and maximum safety.

When properly conducted, the methods described above give a clear primary liquor, without any solids. The primary liquor can then be treated as described below.

The product ion [(FSO₂)₂N]⁻ (“FSI”) can be isolated as one of several metal salts by the method of Morinaka, or alternatively, by removal of the volatile solvent and unreacted base to give a concentrated primary liquor, followed by isolation of an FSI-containing product using a wide variety of organic cationic species, [A]⁺ (i.e., counterions). In some embodiments, a C₁-C₅ tetraalkylammonium halide salt is used, in particular, tetrabutylammonium bromide. The product [Bu₄N]⁺[FSI]⁻ has a melting point of 97-99° C. that enables the solid to be handled under ambient conditions, is insoluble in water, only slightly soluble in cold methanol, and very soluble in hot methanol. Maximum recovery of the FSI anion, by recrystallization, can be achieved this way to give a highly pure, halide-free product. Furthermore, if the concentrated primary liquor is treated directly with a methanolic solution of Bu₄NBr, then chilled and filtered, the filtrate waste is flammable enough to be burned directly. This is a considerable cost saving at large scale. Another symmetric tetraalkylammonium product, [Me₄N]⁺[FSI]⁻ (formed using [Me₄N]⁺[Cl]⁻), gave a product having 10-17 ppm chloride after isolation from the reactor, and undetectable amounts of chloride (<10 ppm by ion chromatography) after a second recrystallization from distilled water. The recovery of FSI as Me₄NFSI is, however, about 10% lower than for Bu₄NFSI. Symmetric alkylammonium salts obtained by this method are of very high purity, and can be dried to very low water levels.

A large number of other organic species [A]⁺ can be used to isolate the FSI anion as a salt. As used herein, a “salt” refers to an association or complex of one or more positively charged species and one or more negatively charged species. In some embodiments, a salt is an ion pair. Any soluble ion pair, ([A]^{x+})ₘ([X]Y⁻)ₙ, where x, y, m, and n are independently integers from 1 to 4, can be added to the crude product to form a new ion pair (e.g., [A]⁺[FSI]⁻) which is either “slightly soluble” (or less) in water (i.e., a solubility of 1% w/v or less), or “soluble” in an organic solvent (e.g., dichloromethane, ethyl acetate, and the like). The counterion [X]⁻ is not important, the only requirement being a counter anion whose salt ([BH]^{x+})ₘ([X]Y⁻)ₙ where x, y, m, and n are independently integers from 1 to 4 (e.g., [X]^{y-}=halide, sulfate, phosphate, acetate, etc, where [BH]⁺ is the protonated nonreactive base B) is more soluble in water than [A]⁺[FSI]⁻.

Cationic species, [A]^{x+}, where x is an integer from 1 to 4, suitable for use in isolating the FSI anion in embodiments of the present invention include the following:

Asymmetric linear or branched alkylammonium species (e.g., butyltrimethylammonium, dimethylethylbutylammonium, trimethyl(3-methylpentyl)ammonium, and alkyl and alkoxyl congeners thereof);

Symmetric and asymmetric pyrrolidinium species (e.g., spirobipyrrolidinium, N-methyl-N-butylpyrrolidinium, N-methyl-N-(2-methoxyethy)pyrrolidinium, and alkyl and alkoxyl congeners thereof);

Symmetric and asymmetric piperidinium species (e.g., spirobipiperidinium, N-methyl-N-butylpiperidinium, N-methyl-N-(2-methoxyethy)piperidinium, and alkyl and alkoxyl congeners thereof);

Symmetric and asymmetric morpholinium species (e.g., spirobimorpholinium, N-methyl-N-butylmorpholinium, N-methyl-N-(2-methoxyethy)morpholinium, and alkyl and alkoxyl congeners thereof);

Symmetric and asymmetric azepinium species (e.g., spirobiazepinium, N-methyl-N-butylazepinium, N-methyl-N-(2-methoxyethyl)azepinium, and alkyl and alkoxyl congeners thereof);

Bicyclic ammonium species (e.g., N-butyl-1-azabicyclooctane, and alkyl and alkoxyl congeners thereof), and alkyl and alkoxyl congeners of other bicyclic ammonium comounds;

Symmetric and asymmetric sulfonium species (e.g., triethylsulfonium, propyldimethylsulfonium, and alkyl and alkoxyl congeners thereof);

Pyridinium species (e.g., N-butylpyridinium and alkyl and alkoxyl congeners thereof);

Imidazolium species (e.g., 1-methyl-3-propylimidazolium, 1-methyl-3-(2-methoxyethyl)imidazolium, and alkyl and alkoxyl congeners thereof);

Biimidazolium, pyrazolium, triazolium, quinolinium species, etc.

Symmetric and asymmetric phosphonium species (e.g., tetramethylphosphonium and symmetric, asymmetric, and wholly or partially alicyclic congeners thereof), which are similar to species outlined herein supra, but with phosphorus instead of nitrogen as the charged atom;

Partially or wholly fluorinated derivatives of any of the above species;

Polycationic congeners of any of the above species, e.g., [(CH₃)₃N(CH₂)₄)N(CH₃)₃]²⁺.

Many of the above choices of counter-cation result in an ionic liquid product. Ionic liquids are well suited to large scale preparation and isolation as the entire workup is all-liquid, with no need to isolate a solid intermediate. FSI ionic liquids have exceptionally low viscosity which makes them suitable for several applications, for example, as neat electrolytes in electrochemical double-layer capacitors, batteries, and as lubricants.

### EXAMPLES

### Example 1

A 600 mL pressure reactor (Parr Instrument Company), equipped with several inlets for pressure measurement and gas introduction, a stirring assembly, and a vacuum gauge, was charged with dry acetonitrile (300 mL) and dry triethylamine (125 grams, 1.23 moles). The reactor was sealed and cooled with stirring to −46° C. and evacuated to 1 torr pressure. Sulfuryl fluoride (SO₂F₂, 18.1 grams, 0.178 mole) was introduced into the reactor and the reactor contents stirred and warmed to 0° C. with a water/ice bath, establishing a static internal pressure of 609 torr. NH₃ gas (2.65 grams, 0.0587 mole) was slowly introduced at a constant rate into the void above the stirred reactor contents over a period of 90 minutes, maintaining at all times a temperature below 2° C. During this time the internal pressure dropped from 609 torr to 51 torr. The addition was halted twice during the 90 minute period, at 45 and 65 minutes, for fifteen minutes each time, to establish the static internal pressure and allow introduced NH₃ to be consumed by the SO₂F₂. It was determined by these static checks that an NH₃ partial pressure of 10 torr was employed during the addition. After the addition of NH₃ was complete, the reactor was stirred for 10 hours, whereupon the temperature rose to +4° C. and the pressure rose to 60 torr.

The reactor was opened and the contents transferred to a 1 liter round bottom flask. The volatile components were removed by rotary evaporation at 55° C./17 torr and the resulting liquor diluted with 150 mL water. A biphasic liquid was produced. The upper layer was decanted off and the lower layer again washed with 150 mL water and decanted. The decanted aqueous washes were combined. The undissolved liquid, a yellow heavy oil, was transferred to a 150 mL beaker, placed on a hotplate stirrer, diluted with 50 mL water, and magnetically stirred. Tetramethylammonium chloride ([Me₄N]⁺[Cl]⁻, 13 grams, 0.12 mole) was added to the stirred beaker contents which were brought to 70° C., producing a clear yellow solution. After cooling in ice, filtration, water wash, and drying in vacuo at 80° C., the product was isolated as a white solid, 6.6 grams, m.p. 289° C. to 291° C. (lit m.p. 286° C. to 288° C.). The filtrates from this first crop were added to the combined decanted aqueous washes producing a copious precipitate from the resultant 400 mL suspension. This was cooled in ice, collected by filtration, washed with water, and dried at 80° C. in vacuo to give a second crop, 6.5 grams, m.p. 285-290° C. Combined yield, 13.1 grams (0.059 mole, 66% based on SO₂F₂).

### Example 2

A two-gallon (7.57 L) stainless steel high pressure reactor (Parr Instrument Company, Moline, Ill. USA) was charged with acetonitrile (3.72 kg) and tetramethyl-1,3-propanediamine (TMPDA, 1.50 kg, 11.5 moles). The reactor was evacuated with medium stirring until a static vacuum of 43-45 torr at 10° C. persisted for at least ten minutes. Sulfuryl fluoride (SO₂F₂) was introduced to the reactor through a pressure-gated dip tube until the setpoint pressure of 760 Torr was achieved. At the end of the addition, a total of 227.5 g SO₂F₂ had been added, and the reactor temperature rose from 11° C. to 14° C. The stir rate was then set to 80% of maximum and NH₃ gas (96 g, 5.63 moles) was added at a constant rate over a three hour period, allowing the temperature to rise to 23° C. to 25° C., then cooling as necessary to maintain this temperature range. SO₂F₂ addition at the setpoint pressure was continuous throughout this time. After the NH₃ addition was complete, SO₂F₂ addition continued until the theoretical weight (1.14 kg, 11.2 moles) had been added. The reactor was then stirred at a reduced rate for ten hours; the pressure dropped from 760 to 123 torr and the temperature from 25° C. to 15° C. during this time.

The reactor contents, a clear, light yellow liquid, were transferred via the dip tube to a large rotary evaporator under reduced pressure and the sealed reactor washed with 1 kg acetonitrile, again through the dip tube. Concentration of the combined liquors at 60 C/150 torr to 60 C/80 torr gave 2.886 kg of a viscous liquid residue, which was added at a constant rate over 14 minutes to a vigorously stirred solution of tetrabutylammonium bromide (2 kg, 6.2 mole) in warm (31° C.) water (10 Kg). The glass receptacles were washed with 3×25 mL methanol and added to the stirred pot. The pot was stirred an additional 20 minutes. The solid so obtained was collected by suction filtration and compressed with a rubber dam. The damp solid (3.245 kg) was taken up in warm methanol (4.93 kg), polish filtered, and cooled to −20° C. The crystalline product was collected by filtration, the cake rinsed twice with chilled methanol, and dried at 45° C. in dynamic vacuum to constant weight. Yield, 1.992 kg (4.71 moles, 84.4%) of a white crystalline product; m.p.=97° C. to 99° C.

A second crop (208.2 g, 0.49 mole, 8.8%), m.p.=97° C. to 99° C., was obtained by concentration of the filtrate. The remaining filtrate was combined with the aqueous residue from the initial isolation of the product and further rotovapped down at 60° C. The solid mass which resulted was separated and recrystallized from methanol as before, yielding a third crop (44.6 g, 0.1 mole, 1.9%), m.p.=97° C. to 99° C. Total yield, 2.245 kg (5.31 moles, 95.1%).

### CONCLUSION

These examples illustrate possible embodiments of the present invention. While various embodiments of the present invention have been described above, it should be understood that these are presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A method comprising:
adding NH₃ to a SO₂F₂ solution in a sealed reactor, in the presence of an organic base, at or below atmospheric pressure, to form a dissolved [(FSO₂)₂N]⁻ anion and a dissolved fluoride anion;
optionally distilling off the solvent; and
isolating a salt containing the [(FSO₂)₂N]⁻ anion.

2. The method of claim 1, wherein the NH₃ is added to the SO₂F₂ solution in a molar ratio of 1:2 to 1.1:2 relative to the SO₂F₂.

3. The method of claim 1, comprising agitating the SO₂F₂ solution at a temperature of −10° C. to 40° C.

4. The method of claim 1, wherein the interior surfaces of the reactor are continuously irrigated or wetted with the liquid contents of the reactor.

5. The method of claim 1, wherein the NH₃ is combined with purified gas from the head space of the reactor prior to addition, and injected into the liquid portion of the reactor contents.

6. The method of claim 1, wherein the SO₂F₂ solution comprises a solvent selected from: acetonitrile, propionitrile, dimethylformamide, dimethylacetamide, N-methylpyrollidinone, tetramethylurea, dimethylpropyleneurea, and a combination thereof.

7. The method of claim 1, wherein the organic base is N,N,N′,N′-tetramethyl-1,2-ethanediamine, N,N,N′,N′-tetramethyl-1,3-propanediamine, and combinations thereof.

8. The method of claim 1, wherein the isolating comprises adding a solution of an organic salt comprising an organic cation, [A]^{x+}, where x is an integer from 1 to 4, that forms a salt, ([A]^{x+})ₘ([(FSO2)2N]⁻)ₙ, where m and n are independently integers from 1 to 4.

9. The method of claim 8, wherein the salt ([A]^{x+})ₘ([(FSO2)2N]⁻⁾ₙ, where x, m, and n are independently integers from 1 to 4, precipitates as a solid, and is collected by filtration.

10. The method of claim 8, wherein the organic salt is dissolved in a biphase-forming solvent, and the salt, ([A]^{x+})ₘ([(FSO₂)₂N]^{y-})ₙ, comprises one layer of the resulting biphasic liquid.
